(19) United States
(12) Patent Application Publication
(10) Pub. No.: US 2020/0179312 A1
(43) Pub. Date: Jun. 11, 2020

(54) ANALOGUES OF CYSTEAMINE AS THERAPEUTIC AGENTS FOR CYSTIC FIBROSIS

(71) Applicant: I.E.R.F.C. EUROPEAN INSTITUTE FOR CYSTIC FIBROSIS RESEARCH, Milano (IT)

(72) Inventors: Luigi Maiuri, Milano (IT); Valeria Villella, Milano (IT); Fabio Borella, Milano (IT); Giorgio Cozza, Milano (IT); Andrea Venerando, Milano (IT)

(73) Assignee: I.E.R.F.C. EUROPEAN INSTITUTE FOR CYSTIC FIBROSIS RESEARCH, Milano (IT)

(21) Appl. No.: 16/691,647

(22) Filed: Nov. 22, 2019

(30) Foreign Application Priority Data
Nov. 23, 2018 (EP) 18208109.1

(51) Int. Cl. A61K 31/145; A61P 11/00

(52) U.S. Cl. CPC A61K 31/145; A61P 11/00

(57) ABSTRACT

The present invention concerns a method of treatment of cystic fibrosis in a patient in need thereof, the method comprising administering an effective amount of a compound of formula I

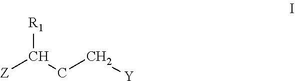

Wherein R1, X. Y and Z are as defined in the description.

This U.S. utility application claims priority to and the benefit of European Application No. 18208109.1 filed Nov. 23, 2018, the content of which is incorporated herein by reference in its entirety.

The present invention concerns a method of treatment of cystic fibrosis in a patient in need thereof, the method comprising administering an effective amount of analogues of cysteamine of formula I.

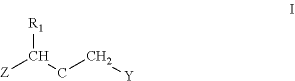

### BACKGROUND OF THE INVENTION

Cystic Fibrosis (CF) is the most common life-shortening rare disease in Caucasians [1], mainly characterized by lung disease, the principal cause of morbidity and mortality, including airway obstruction by viscous mucus and inflammation with recurrent bacterial infections and colonization [2]. CF is caused by mutations in the gene coding for CF Transmembrane Conductance Regulator (CFTR), a protein functioning as a chloride channel at the apical membranes of epithelial cells [3]. CFTR is composed by several domains, two membrane-spanning (MSD-1/2), two ATPase (NBD-1/2), and a regulatory (RD) whose phosphorylation regulates channel gating.

To date, the clinical management of CF has failed to address its primary cause, namely the loss-of-function of CFTR. More than 2000 mutations have been identified in the CFTR gene and then categorized in 6 different classes according to their functional impact [4, 5]. Mutation-directed approaches aimed at correcting the CFTR defect have recently emerged [1, 6]. They focus on the identification of molecules that directly target mutant CFTR, either capable of correcting the trafficking of CFTR mutants (correctors) or improving channel function (potentiators). A mutation-directed CFTR repairing therapy with a channel-potentiator (Ivacaftor) is available for ˜5% of CF patients bearing membrane-resident class III mutant (G551D) [7]. In contrast, for the vast majority (70-90%) of CF patients bearing the most common class II F508del mutation, current mutation-therapy partially corrects the defect [8, 9]. F508delCFTR is retained in the endoplasmic reticulum (ER) and degraded before it reaches the plasma membrane (PM) [10]. Notably that F508delCFTR acquires some degree of function if rescued and stabilized at the PM [11, 12] and restoring about 20-30% of CFTR function is believed to confer at least partial clinical benefit to CF patients [13, 14]. However, when Ivacaftor is combined in vitro with CFTR correctors, the former counteracts the latter by further decreasing F508delCFTR PM stability after rescue [15, 16], thus demonstrating that a novel rationale is needed based on mechanistic rather than mutation-directed approaches.

Alternative strategies, aimed at targeting the cellular environment perturbed by the lack of a functional CFTR instead of directly targeting the mutant CFTR protein, have been recently proposed [17, 18]. In particular autophagy, the major mechanism determining cytoplasmic protein turnover, is blocked due to tissue transglutaminase (TG2)-mediated depletion of the essential autophagy-related protein Beclin 1 (BECN1), leading to secondary accumulation of the autophagic substrate SQSTM1/p62 [19]. Notably that TG2 is a versatile multifunctional protein that acts as a crosslinking enzyme in the presence of high Ca²⁺ levels, and functions as G-protein or protein disulfide isomerase (PDI) at low Ca²⁺ concentrations.

Cysteamine has been proposed as a therapeutic agent in view of its ability to restore CFTR function in patients with cystic fibrosis carrying the F508del-CFTR mutation [20, 21]. EP2664326 disclosed a combination of inhibitors of Tissue Transglutaminase (TG2), inhibitors of reactive oxygen species (ROS) such as cystamine or cysteamine and CFTR channel activators for the treatment of CF patients carrying the F508-CFTR mutation. The ability of cysteamine to inhibit the PDI activity of TG2 was also recently confirmed [22]. As demonstrated before [20], cysteamine is able to restore in vitro the 78% of CFTR function at 250 μM concentration (see Table 1).

CA1157774A discloses the use of several compounds able to release alkylthiolamines for the treatment of cystic fibrosis. The compounds are mucolytic agents and their activity against CFTR is not mentioned.

References [23] and [24] disclose the use of cysteamine in the treatment of cystic fibrosis, whereas, reference [25] discloses the reduction of sputum viscosity in cystic fibrosis patients using amifostine.

### BRIEF DESCRIPTION OF THE DRAWINGS

FIG. 1 shows SPQ assay in F508delCFTR homozygous bronchial epithelial cell lines (CFBE41o-) transfected with F508delCFTR.

FIGS. 2A and 2B show SPQ assay in primary nasal cells.

FIG. 3 shows CFTR function by SPQ assay in CFBE cells.

FIG. 4 shows CFTR function by SQP assay in nasal primary cells.

FIG. 5 shows immunoblots of CFBE cells.

FIG. 6 shows immunoblots of nasal primary cells.

FIG. 7 shows the results of titration of IE1 on CftrF508del mice administered intraperitoneally d with cysteamine or different concentrations of IE1.

FIG. 8 shows immunoblots of lung and small intestinal tissue of CftrF508del mice administered intraperitoneally with cysteamine.

FIG. 9 shows CFTR function of in lung and small intestinal tissue of CftrF508del mice administered intraperitoneally with cysteamine.

FIG. 10 shows the effect of IE1 on whole homogenate from intestinal and lung tissues CftrF508del mice.

FIG. 11 shows the effect of IE1 on whole homogenate from intestinal and lung tissues of CftrF508del mice.

FIG. 12 shows the effect of IE1 on whole homogenate from intestinal and lung tissues of CftrF508del mice.

FIG. 13 shows results on inflammation induced by IE1 on whole homogenate from intestinal and lung tissues of CftrF508del mice.

### DESCRIPTION OF THE INVENTION

It has now been found that cysteamine analogues of formula I are particularly effective in restoring a functional F508delCFTR protein and improving disease phenotype. In addition, the compounds of formula I do not only directly target the F508delCFTR protein, but also other proteins whose malfunction is causal of the low CFTR expression at the cell surface, in particular TG2 and its PDI activity.

The invention accordingly provides a method of treatment of cystic fibrosis in a patient in need thereof, the method comprising administering an effective amount of a compound of formula I

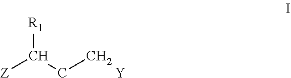

wherein:
Z is —NH₂ or NH₂—NH—, preferably —NH₂;
R1 is H, C₁-C₃-alkyl, phenyl optionally substituted by methyl or ethyl, 2-, 3- or 4-pyridyl;
X is a divalent group of formula —(CH₂)ₙ—, C═O, —NH—, —S—, —(CH₂)ₘ—NH—(CH₂)ₘ— or —S—(CH₂)ₚ-n is zero or an integer from 1 to 4, m is 1 or 2, o is 1 or 2 and p is 1 or 2;
Y is —SH, —SeH or —SPO₃H (phosphorothioate);
provided that the compound of formula I is not cysteamine or 2-(3-aminopropyl)aminoethyl phosphorothioate (amifostine).

In a first preferred embodiment, the method comprises administering an effective amount of a compound of formula I wherein Z is —NH₂, R1 is hydrogen, phenyl optionally substituted by methyl or ethyl, 2-, 3- or 4-pyridyl, X is —(CH₂)ₙ— and n is zero when R1 is different from hydrogen or n is 1 to 3 and Y is —SH or —SPO₃H.

In a second preferred embodiment, the method comprises administering an effective amount of a compound of formula I wherein Z is —NH₂, R1 is hydrogen, X is C═O or —(CH₂)ₙ— wherein n is from 1 to 4 and Y is —SH.

In a third preferred embodiment, the method comprises administering an effective amount of a compound of formula I wherein Z is —NH₂ or NH₂—NH—, R1 is hydrogen, X is —NH— or —(CH₂)ₘ—NH—(CH₂)ₒ— wherein m and o are independently 1 or 2 and Y is —SH or —SPO₃H.

In a fourth preferred embodiment, the method comprises administering an effective amount of a compound of formula I wherein Z is —NH₂, X is —S— or —S—(CH₂)ₚ— and Y is —SH.

Preferred compounds of formula I to be administered in the method include 3-aminopropane-1-thiol, 4-aminobutane-1-thiol, 5-aminopentane-1-thiol, and 6-amino-1-hexanethiol. 3-Aminopropane-1-thiol is particularly preferred.

The compounds of formula I are either known or may be prepared according to conventional synthetic methods. For the intended therapeutic method, the compounds of the invention will be administered by any suitable route, e.g. by the oral, parenteral or aerosol route. Suitable compositions comprising an effective amount of compounds of formula I and optionally other active ingredients in combination with other pharmaceutically acceptable excipients will be prepared according to known techniques. Examples of said compositions include tablets, capsules, syrups, solutions, suspensions.

The dose of compounds of formula I will be adapted to the specific situation taking into account patient's age, general health conditions, weight, other concomitant therapies and responsiveness to the combined treatment over time. A range of 10 to 1000 mg/dose once or more times a day will be usually acceptable.

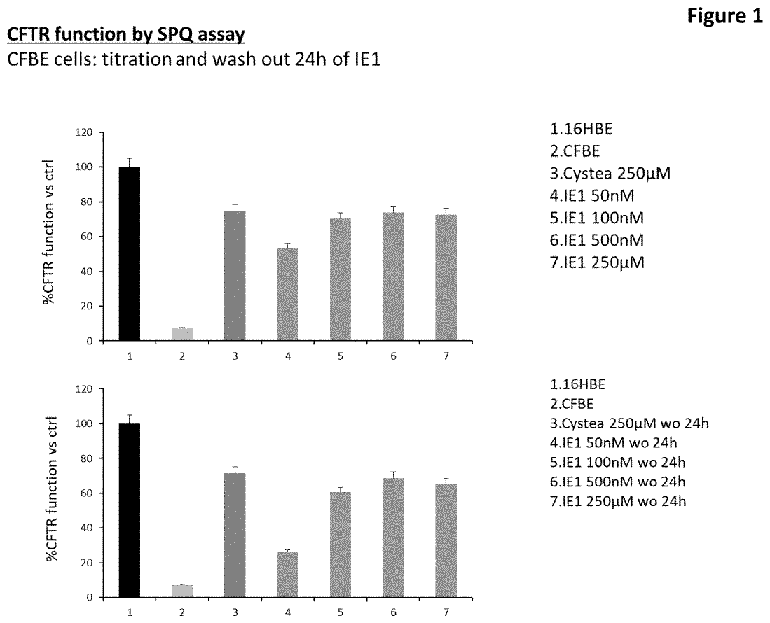

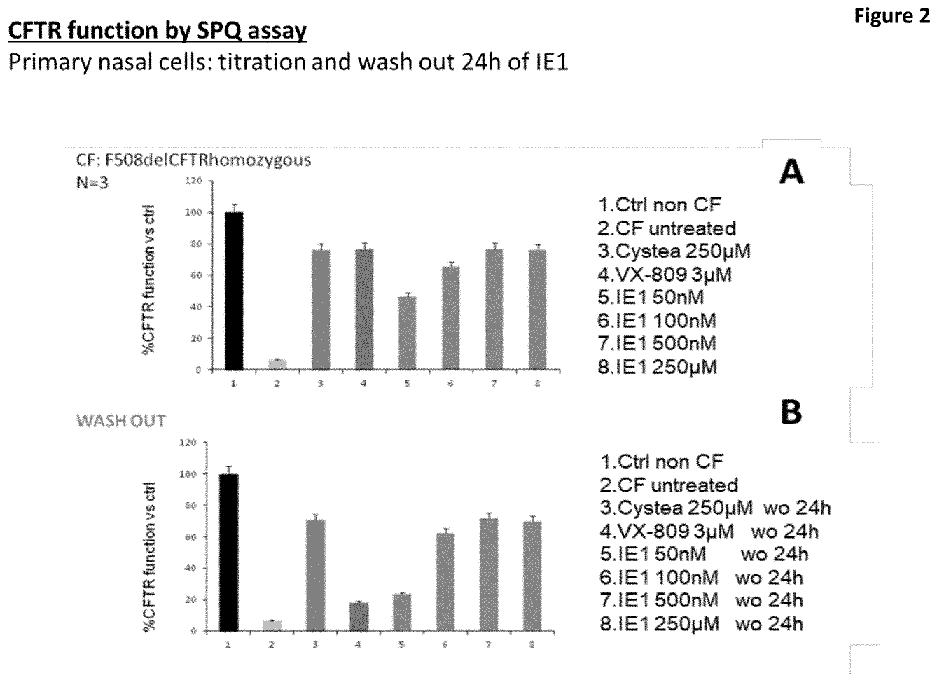

The compounds of formula I have marked effects on CFTR function. A representative number of compounds of formula I (Compounds IE1-IE4; Table I) were tested in comparison to cysteamine and amifostine through SPQ assay in F508delCFTR homozygous bronchial epithelial cell lines (CFBE41o-) transfected with F508delCFTR (FIG. 1) and in ex vivo in nasal cells collected by nasal brushing from CF patients (FIGS. 2A and B).

TABLE 1
Effect on F508delCFTR function testes via SPQ in
bronchial epithelial cell lines (CFBE41o-) transfected
with F508delCFTR. 16HBE are used as control.

| | | Concen- | |
|---|---|---|---|
| Compound | | tration | % Function vs |
| Name | IUPAC Name | (μM) | 16HBE |
| Cysteamine | 2-aminoethane-1-thiol | 250 | 78% |
| IE1 | 3-aminopropane-1-thiol | 0.1 | 72% |
| IE2 | 4-aminobutane-1-thiol | 0.5 | 71% |
| IE3 | 5-aminopentane-1-thiol | 0.5 | 59% |
| IE4 | 6-amino-1-hexanethiol | 100 | 69% |
| Amifostine | 2-(3-aminopropyl)aminoethyl | 50 | 58% |
| | phosphorothioate | | |

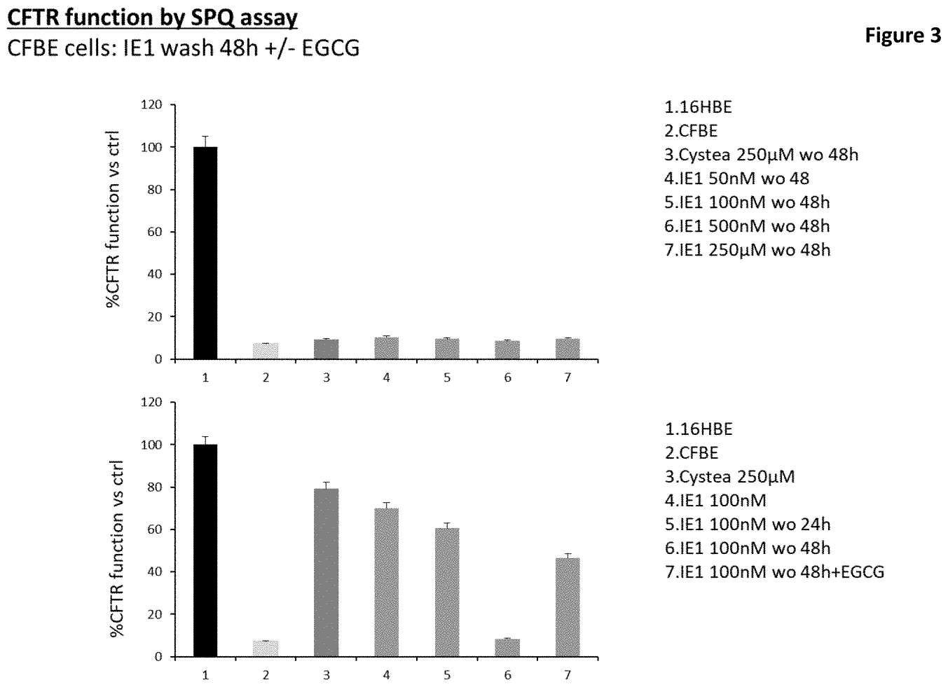

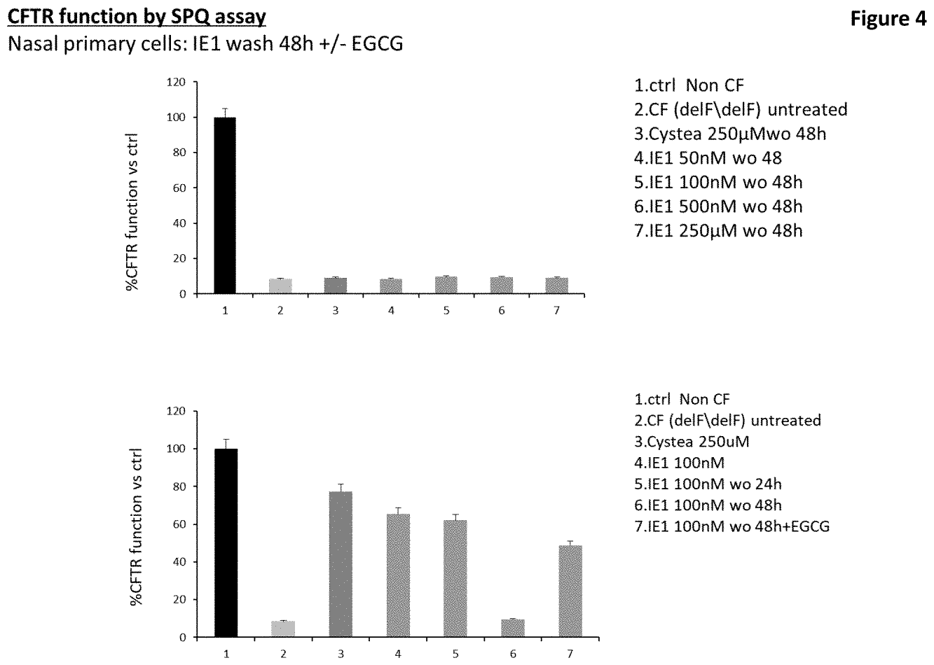

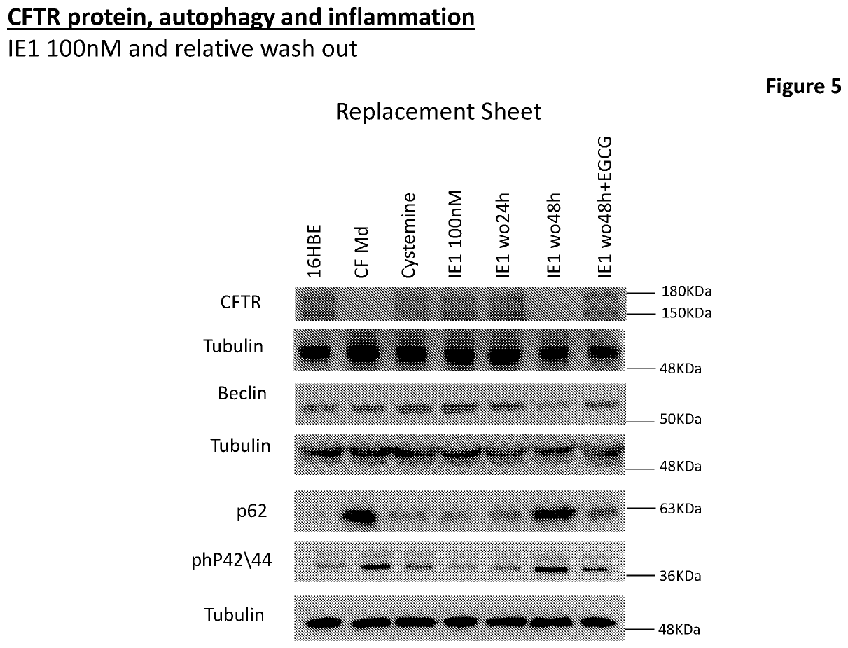

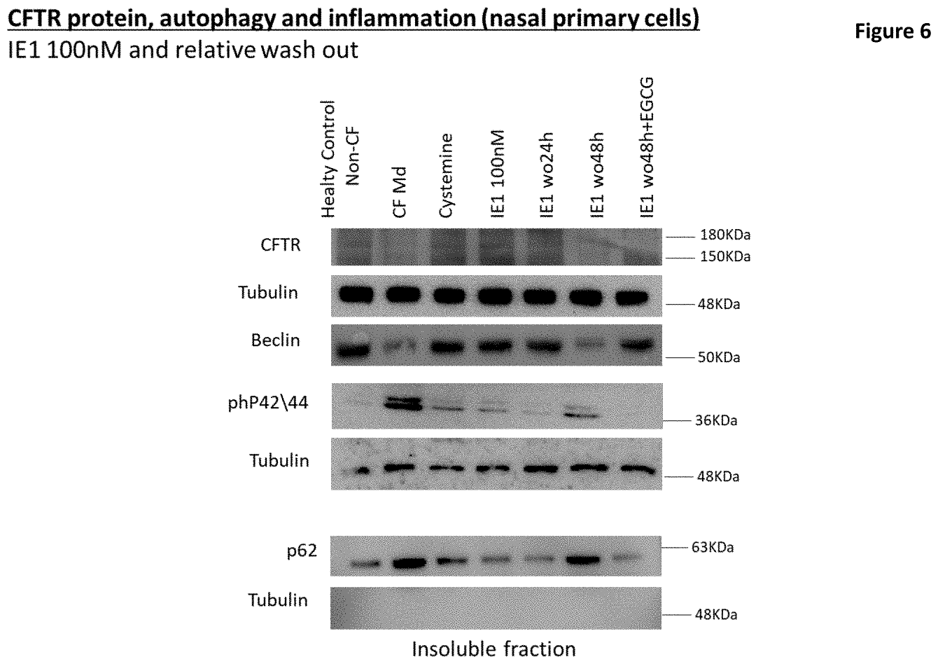

By comparing cysteamine with IE1 it is evident that IE1 is able to restore CFTR function equally to cysteamine, but at a concentration more than 2500 fold lower (0.1 μM vs 250 μM). IE1 has been demonstrated more efficacious than VX-809 (3 μM, FIG. 2A). Moreover, as demonstrated for cysteamine, IE1 is able to maintain CFTR function up to 24 h after wash out, a result not obtained by the corrector VX-809 (See FIGS. 1B and 2B). To note that IE1 loses its efficacy after 48 h after wash out (FIGS. 3 and 4), however its effect is preserved by adding epigallocatechin gallate (EGCG, FIGS. 3 and 4), an effect previously demonstrated for cysteamine [20]. According to the results obtained with IE1 on CFTR function, immunoblot detection of CFTR confirms that a sizeable fraction of the channel resided at the PM after treatment with 0.1 μM IE1 (compared with 250 μM cysteamine) and maintained after 24 h of the compound washout. As expected by the function experiments, the amount of PM-resident CFTR protein decreases after 48 h of washout but is completely maintained when EGCG is added to the experiment. These results were obtained in both CFBE cells (FIG. 5) and ex vivo cells collected by nasal brushing (FIG. 6). As in the case of cysteamine, the ability of IE1 to inhibit the PDI activity of TG2 was also confirmed.

All of the tested compounds are able to restore more than 50% of CFTR function.

IE1 Effect on Authophagy and Inflammation

The restoration of Beclin 1-dependent autophagy and the depletion of SQSTM1/p62 by genetic intervention or chemical modulators have positive effects on the CFTR function both in cell and in vivo. As shown in the case of cysteamine, immunoblot analysis shows that IE1 (0.1 μM) is able to restore BECN1 and to reduce p62 levels with the consequent improvement of CFTR function (see above). This effect disappears after 48 h but is preserved by EGCG addition. In addition, the inflammation biomarkers (phospho)p42/44 MAPK decrease in the presence of IE1 (0.1 μM), an effect completely lost after 48 h but maintained in presence of EGCG. These results were obtained by treating both CFBE cells (FIG. 5) and cells collected ex vivo by nasal brushing (FIG. 6).

Mouse Model

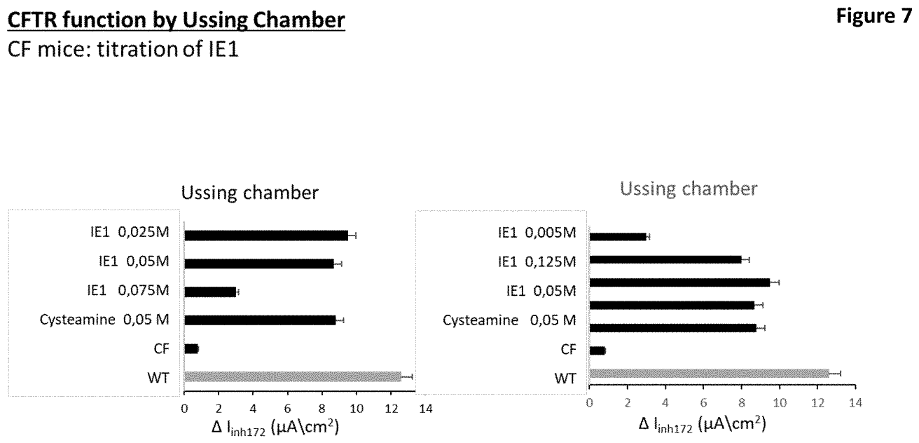

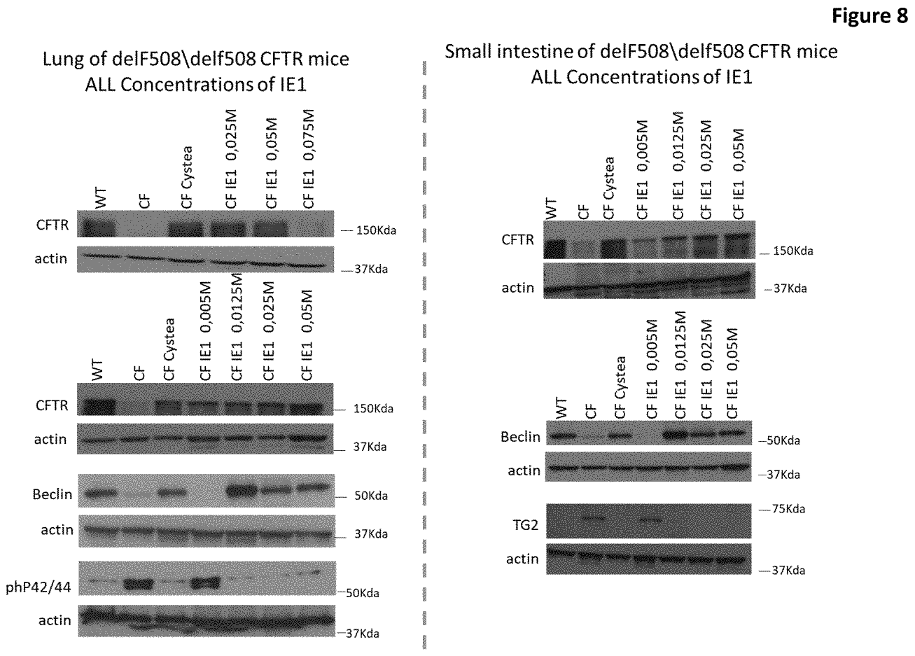

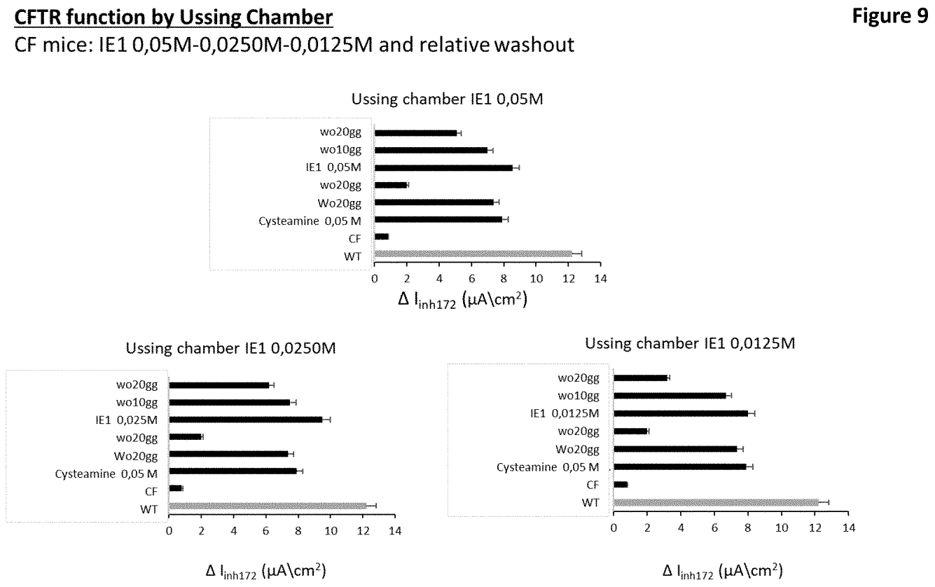

Driven by the positive outcome of the cell lines (immortalized and primary nasal cell) experiments, we next investigated the possibility that IE1 could rescue the CFTR protein and function as well as re-establish the autophagy pathway in CF mice model. To test this hypothesis, we used F508del-Cftr homozygous (CftrF508del\F508del) mice and control mice (Cftr WT). CftrF508del mice were administered intraperitoneally (i.p.) for 5 d with cysteamine or different concentrations of IE1 (FIG. 7). As shown, the Ussing chamber experiments (on small intestinal tissues) revealed that the range concentration of IE1 from 0.05 to 0.00125 is highly effective on rescue the CFTR function. Next, CftrF508del mice were administered intraperitoneally (i.p.) for 5 d with cysteamine or with 3 different concentrations of IE1 (0.05M, 0.025M or 0.0125M) followed by 10 d or 20 d of vehicle only (FIG. 9). In this experimental model, the effects of cysteamine or IE1 in sustaining the CFTR function highlighted that all of three concentrations of IE1 are more effective than cysteamine to sustain the effect after 20 d of wash out. The immunoblot analysis of CFTR protein levels confirms the effect of IE1 on sustaining the protein well beyond 20 d of wash out (FIG. 8-9).

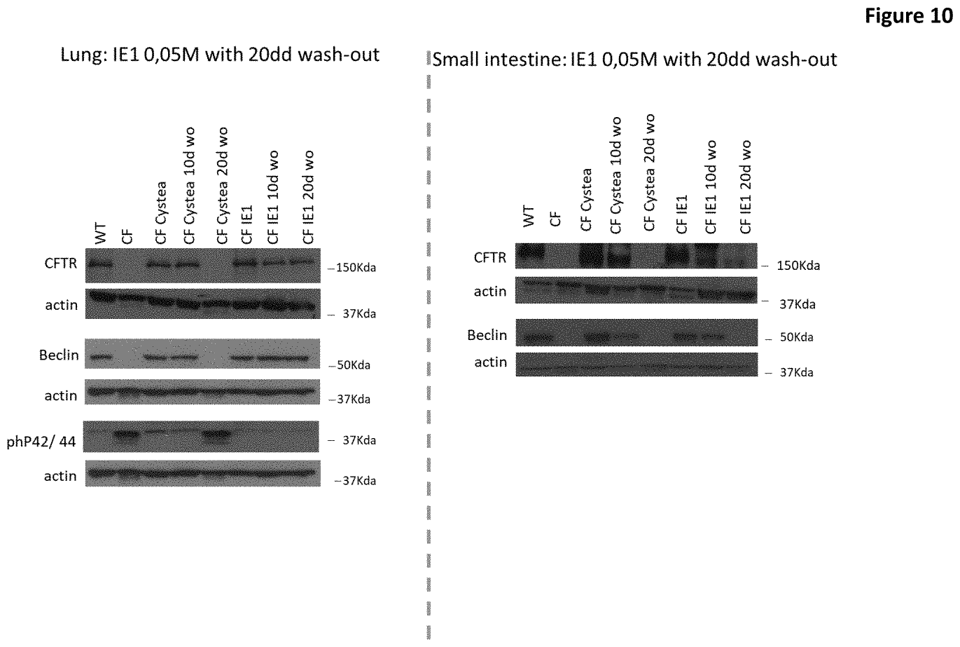

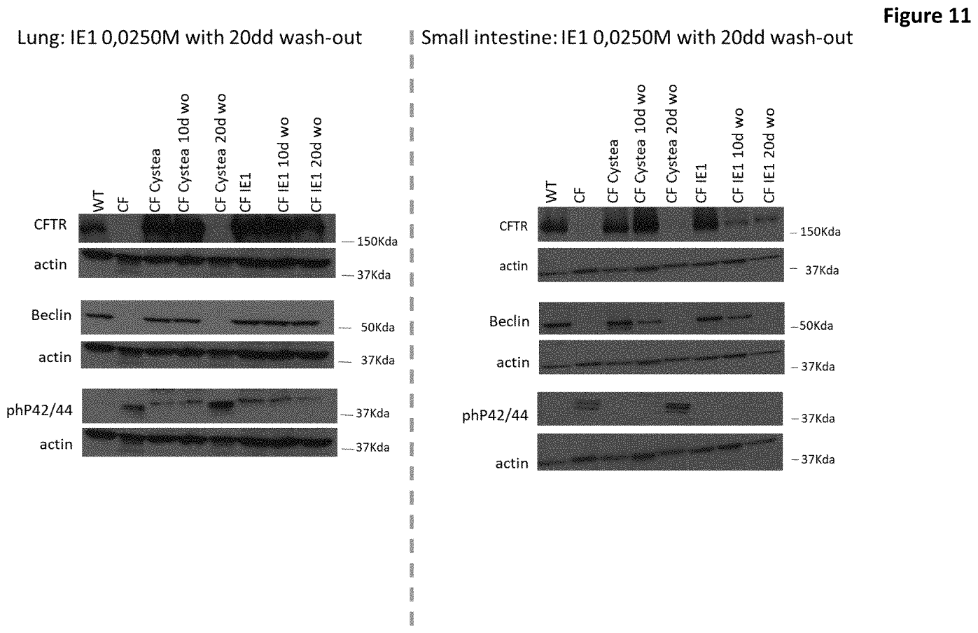

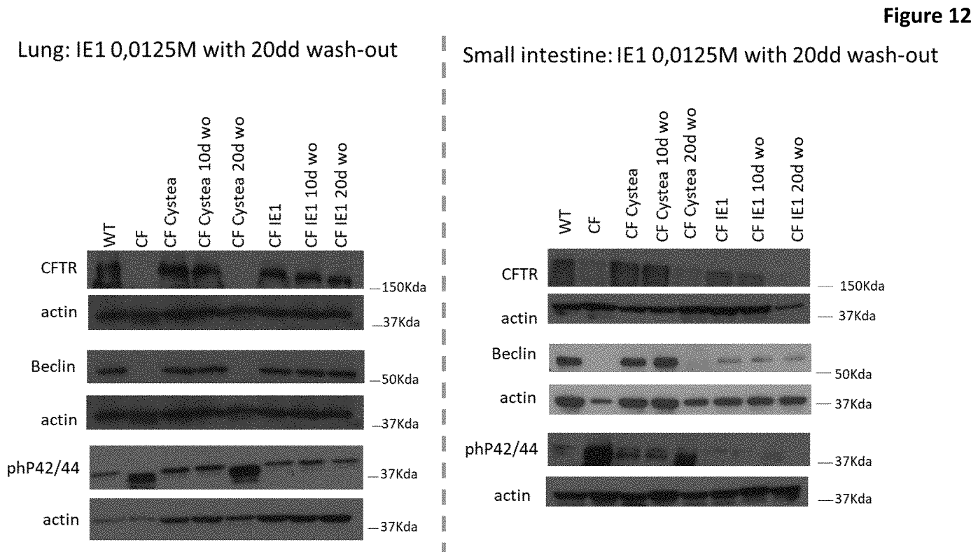

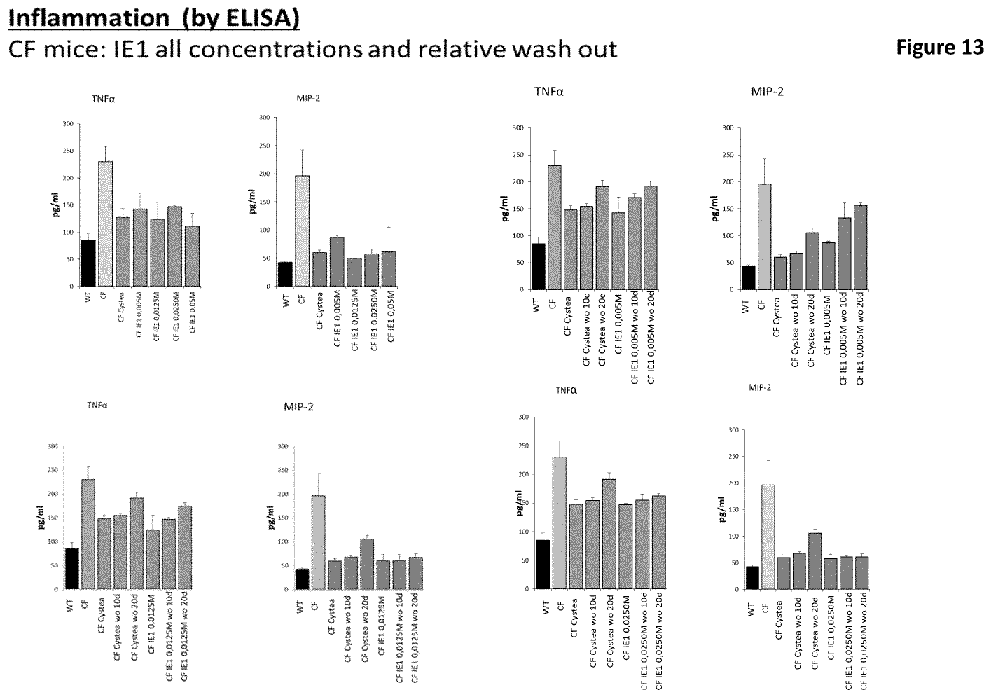

To confirm the effect of IE1 by acting on autophagy and inflammatory pathway, immunoblot analysis of whole homogenates from intestinal and lung tissues showed that IE1 concentrations are able to restore BECN1 and decrease biomarkers (phospho)p42/44 MAPK (FIG. 10-12), and decrease principal inflammatory cytokines (i.e TNF alpha and MIP-2) (FIG. 13).

Methods

Animal Tests.

CF mice homozygous for the delF508-CFTR mutation (abbreviated CftrF508del) in the 129/FVB outbred background (CftrtmlEUR, F508del, FVB/129) were obtained from Erasmus Medical Center Rotterdam, The Netherlands (CF coordinated action program EU FP6 LSHM-CT-2005-018932). These studies and procedures were approved by the local Ethics Committee for Animal Welfare (IACUC No 713). Young adult CF mice and their wild-type (wt) littermates were housed in static isolator cages at the animal care specific pathogen-free facility of Charles River (Calco, Italy). CftrF508del mice were treated with daily intraperitoneal injections of cysteamine (100 μl of 0.05 M in PBS) (Sigma Aldrich, M9768) or IE1 (100 μl of 0.075-0.05-0.025-0.0125 M, in PBS) for 5 d or with cysteamine (5 d) or IE1 different concentrations followed by 10 d or 20 d of PBS alone (n=5 mice per group) (19,20).

Cell lines experiments. CFBE41l- and 16HBE14o-cells were cultured in Minimum Essential Medium (MEM) with Earle's salt, 200 mM L-Glutamine, 10% FBS and penicillin/streptomycin. CFBE41o-cells were transfected with AF508-CFTR at 37° C. and incubated for 18 h with cysteamine (250 μM) or different concentration of IE1.

Nasal Brushing.

Freshly isolated brushed nasal epithelial cells were collected by nasal brushing, as previously described (21, 21) from 10 F508del-CFTR homozygous CF patients and from 5 non-CF healthy volunteers recruited from staff at the same time points. Both patients and healthy volunteers gave written informed consent. After nostril washing to remove mucus, cytological brushes (Robimpex, MO11157) were used to scrape the mid part of the inferior turbinate from both nasal nostrils. Brushes with cells were immediately transferred into RPMI 1640 medium (Invitrogen) containing 1% penicillin-streptomycin (Lonza, 17-602E), in 15-ml sterilized tubes. The tubes were incubated at 37° C. for 2 h on a thermo shaker, to remove all cell from brushes; the brushes were then removed and the cells centrifuged at 800×g (2000 rpm) for 20 min. The supernatant fractions were discarded and the cell pellet treated with 150 mL of trypsin-versene (EDTA) solution (Lonza, 17-161) for 4 min at 37° C. to disaggregate possible cell clusters. Trypsin solution was inactivated by adding 3 ml of serum-free Bronchial Epithelial cell Growth Medium BEGM (Clonetics, Lonza, CC3170). After centrifugation at 800×g (2000 rpm) for 10 min, cells were placed in CELLC T 25 flasks (Sarstedt Ltd, CS300) with 10 ml of BEGM medium. Nonspecific epithelial cells were removed during the daily cell washing and medium changes.

Immunoblot Analysis.

The proteins of cell lines lysates or lung or small intestine homogenates, obtained from treated and untreated cells or mice, were quantified by a Bio-Rad protein assay to ensure equal protein loading before immunoblot analysis. Fifty micrograms of proteins were loaded in each lane. Antibodies against SQSTM1, (Sigma Aldrich,P0067) 1:1000, BECN1 (Abcam, ab58878), 1:1000, CFTR clone CF3 (Abcam, ab2784) 1:1000 or anti-CFTR antibody 660 1:1000, Phospho-p44/42 MAPK (Erk1/2) (Thr202/Tyr204) (Cell Signaling Technology, #91101) 1:1000, TG2 (CUB7402, NEOMARKER) 1:1000 were used. Normalization was performed by probing the membrane with anti-β-actin (Cell Signaling, #4970) 1:1000 or anti-α-tubulin (Cell Signaling Technology, 2148) 1:1000.

ELISA. ELISA was performed on tissue samples using standard ELISA kits (R&D Systems) for TNF-α, Mip2a. According to the manufacturer's instructions, samples were read in triplicate at 450 nm in a microplate Reader (Bio-Rad, Milan, Italy) using Microplate Manager 5.2.1 software.

Values were normalized to protein concentration evaluated by Bradford analysis.

### REFERENCES

[1] K. DE BOECK ET AL., J CYST FIBROS, 13 (2014) 403-409.
[2] F. RATJEN ET AL., CYSTIC FIBROSIS, LANCET, 361 (2003) 681-689.
[3] J. R. RIORDAN ET AL., SCIENCE, 245 (1989) 1066-1073.
[4] M. D. AMARAL ET AL., CURR PHARM DES, 19 (2013) 3497-3508.
[5] M. D. AMARAL ET AL., TRENDS PHARMACOL SCI, 28 (2007) 334-341.
[6] M. D. AMARAL, J INTERN MED, 277 (2015) 155-166.
[7] B. W. RAMSEY, ET AL., N ENGL J MED, 365 (2011) 1663-1672.
[8] A. M. JONES ET AL., THORAX, 70 (2015) 615-616.
[9] M. P. BOYLE ET AL., LANCET RESPIR MED, 2 (2014) 527-538.
[10] C. M. FARINHA ET AL., FEBS J, 280 (2013) 4396-4406.
[11] G. L. LUKACS ET AL., J BIOL CHEM, 268 (1993) 21592-21598.
[12] G. L. LUKACS ET AL., TRENDS MOL MED, 18 (2012) 81-91.
[13] E. KEREM, CURR OPIN PULM MED, 10 (2004) 547-552.
[14] A. S. RAMALHO ET AL., AM J RESPIR CELL MOL BIOL, 27 (2002) 619-627.
[15] D. M. CHOLON ET AL., SCI TRANSL MED, 6 (2014) 246RA296.
[16] G. VEIT, R. G. ET AL., SCI TRANSL MED, 6 (2014) 246RA297.
[17] W. E. BALCH, D. M. ROTH, D. M. HUTT, EMERGENT PROPERTIES OF PROTEOSTASIS IN MANAGING CYSTIC FIBROSIS, COLD SPRING HARB PERSPECT BIOL, 3 (2011).
[18] S. PANKOWET AL., NATURE, 528 (2015) 510-516.
[19] A. LUCIANI ET AL., NAT CELL BIOL, 12 (2010) 863-875.
[20] D. DE STEFANO ET AL., AUTOPHAGY, 10 (2014) 2053-2074.
[21] A. TOSCO ET AL., CELL DEATH DIFFER, 23 (2016) 1380-1393.
[22] F. ROSSIN ET AL., EMBO REP, (2018).
[23] S. ZHANG ET AL., CELL DEATH & DISEASE, 9(2) (2018) 191.
[24] G. DEVEREUX ET AL., CLINICAL DRUG INVESTIGATION, 36 (2016) 605-612.
[25] N. F. TABACHNIK ET AL., JOURNAL OF PHARMACOLOGY AND EXPERIMENTAL THERAPEUTICS, 214 (1980) 246-249.

1. A method of treatment of cystic fibrosis in a patient in need thereof, the method comprising administering to said patient an effective amount of a compound of formula I 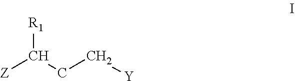
wherein:
Z is —NH₂ or NH₂—NH;
R1 is H, C₁-C₃-alkyl, phenyl optionally substituted by methyl or ethyl, 2-, 3- or 4-pyridyl;
X is a divalent group of formula —(CH₂)ₙ—, C═O, —NH—, —S—, —(CH₂)ₘ—NH—(CH₂)ₒ— or —S—(CH₂)ₚ—;
n is zero or an integer from 1 to 4, m is 1 or 2, o is 1 or 2 and p is 1 or 2;
Y is —SH, —SeH or —SPO₃H;
provided that the compound of formula I is not cysteamine or 2-(3 aminopropyl)aminoethyl phosphorothioate (amifostine).

2. The method of claim 1 wherein Z is —NH₂, R1 is hydrogen, phenyl optionally substituted by methyl or ethyl, 2-, 3- or 4-pyridyl, X is —(CH₂)ₙ— and n is zero when R1 is different from hydrogen or n is 1 to 3 and Y is —SH or —SPO₃H.

3. The method of claim 1 wherein Z is —NH₂, R1 is hydrogen, X is C═O or —(CH₂)ₙ— wherein n is from 1 to 4 and Y is —SH.

4. The method of claim 1 wherein Z is —NH₂ or NH₂—NH—, R1 is hydrogen, X is —NH— or —(CH₂)ₘ—NH—(CH₂)ₒ— wherein m and o are independently 1 or 2 and Y is —SH or —SPO₃H.

5. The method of claim 1 wherein Z is —NH₂, X is —S— or —S—(CH₂)ₚ— and Y is —SH.

6. The method of claim 1 wherein the compound is selected from 3-aminopropane-1-thiol, 4-aminobutane-1-thiol, 5-aminopentane-1-thiol, and 6-amino-1-hexanethiol.

7. The method of claim 1 wherein the compound is 3-aminopropane-1-thiol.

8. The method of claim 1, wherein Z is —NH₂.
